(19) United States
(12) Patent Application Publication
(10) Pub. No.: US 2023/0057671 A1
(43) Pub. Date: Feb. 23, 2023

(54) VERTICAL-FLAT-ARRANGEMENT-TYPE SUCTION AND IRRIGATION DEVICE

(71) Applicant: ORANGE MEDICS, INC., Daejeon (KR)

(72) Inventor: Ki Hwan LEE, Daejeon (KR)

(73) Assignee: ORANGE MEDICS, INC., Daejeon (KR)

(21) Appl. No.: 17/982,547

(22) Filed: Nov. 8, 2022

Related U.S. Application Data
(63) Continuation of application No. PCT/KR2021/009311, filed on Jul. 20, 2021.

(30) Foreign Application Priority Data
Nov. 18, 2020 (KR) 10-2020-0154370

(51) Int. Cl. A61M 1/00

(52) U.S. Cl. CPC A61M 1/743; A61M 1/86; A61M 1/774

(57) ABSTRACT

Disclosed herein is a vertical-flat-arrangement-type suction and irrigation device which is easy to operate. The vertical-flat-arrangement-type suction and irrigation device includes a housing, a probe, and a suction and supply unit. The housing include a front chamber and a rear chamber spaced apart from each other and connected via a connection duct, a first duct connected to a front of the front chamber and extending forwards, a second duct connected to a rear of the front chamber and extending rearwards, and a third duct connected to a rear of the rear chamber and extending rearwards. The suction and supply unit includes a supply valve reciprocally moved inside the front chamber in an axial direction of the front chamber and a suction valve reciprocally moved inside the rear chamber in an axial direction of the rear chamber.

### TECHNICAL FIELD

The present invention relates to a vertical-flat-arrangement-type suction and irrigation device and, more particularly, to a vertical-flat-arrangement-type suction and irrigation device which is easy to operate.

### BACKGROUND ART

In general, endoscopic (or laparoscopic) surgery is minimally invasive surgery and has been rapidly developing in recent years due to advantages thereof, such as less pain, less infection, minimal incision, and reduced hospital stay.

Laparoscopic surgery uses a surgical instrument for supplying an irrigation solution for irrigation of a surgical site of a patient and removing contaminated fluids after irrigation of the surgical site through discharge of the contaminated fluids from the patient's body, that is, a laparoscopic suction and irrigation system.

A typical laparoscopic suction and irrigation system includes a probe inserted into a patient's body, a main body having a mixing chamber connected to the probe, an irrigation channel connected to the mixing chamber to supply an irrigation fluid into the patient's body therethrough, and a discharge channel through which contaminated fluids are discharged from the patient's body.

Although various types of laparoscopic suction and irrigation systems are being put into use, there is a continuous need for an easy-to-operate laparoscopic suction and irrigation system.

As an example of prior documents, there is Korean Patent Registration No. 1837055 (published on Mar. 9, 2018).

### DISCLOSURE

### Technical Problem

Embodiments of the present invention are conceived to solve such problems in the art and it is an object of the present invention to provide a vertical-flat-arrangement-type suction and irrigation device which is easy to operate.

It should be understood that the present invention are not limited to the above. The above and other objects of the present invention will become apparent to those skilled in the art from the detailed description of the following embodiments in conjunction with the accompanying drawings

### Technical Solution

In accordance with one aspect of the present invention, a vertical-flat-arrangement-type suction and irrigation device includes: a housing having a front chamber and a rear chamber spaced apart from each other and connected via a connection duct, a first duct connected to a front of the front chamber and extending forwards, a second duct connected to a rear of the front chamber and extending rearwards, and a third duct connected to a rear of the rear chamber and extending rearwards; a probe coupled to the first duct and inserted into a patient's abdominal cavity; and a suction and supply unit having a supply valve reciprocally moved inside the front chamber in an axial direction of the front chamber, the supply valve forming a front fluid passage connected to the first duct and allowing an irrigation fluid to be delivered to the probe through the first duct, and a suction valve reciprocally moved inside the rear chamber in an axial direction of the rear chamber.

In one embodiment, the supply valve may include a supply valve body spaced apart from an inner peripheral surface of the front chamber and a pair of flanges protruding from opposite ends of the supply valve body and closely contacting the inner peripheral surface of the front chamber, and the front fluid passage may be an inner space defined by an outer peripheral surface of the supply valve body, the inner peripheral surface of the front chamber, and the pair of flanges, the front fluid passage being formed inside the front chamber at all times and being moved in conjunction with movement of the supply valve.

In one embodiment, the supply valve may further include a sealing ring closely contacting outer peripheral surfaces of the pair of flanges and the inner peripheral surface of the front chamber to seal the front fluid passage.

In one embodiment, the supply valve may further include a first resilient member disposed inside the front chamber and having a lower end supported by the front chamber and an upper end supported by the supply valve to resiliently support the supply valve so as to disconnect the front fluid passage from the second duct.

In one embodiment, the suction valve may include a suction valve body spaced apart from an inner peripheral surface of the rear chamber, a sliding block protruding from the suction valve body and moved together with the suction valve body, and a stationary sealing ring secured to an upper portion of the rear chamber and closely contacting an outer peripheral surface of the suction valve body, and, when the suction valve is moved downwards, a rear fluid passage defined by the outer peripheral surface of the suction valve body, the inner peripheral surface of the rear chamber, the stationary sealing ring, and an upper portion of the sliding block may be formed, the rear fluid passage being connected to the connection duct and the third duct.

In one embodiment, the suction valve may further include a sealing plate disposed on the sliding block and closely contacting the inner peripheral surface of the rear chamber, the sealing plate closing the connection duct and the third duct upon upward movement of the suction valve body while opening the connection duct and the third duct upon downward movement of the suction valve body.

In one embodiment, the suction valve may further include a second resilient member disposed inside the rear chamber and having a lower end supported by the rear chamber and an upper end supported by the suction valve to resiliently support the suction valve such that the connection duct and the third duct are closed by the sealing plate.

In one embodiment, the connection duct and the third duct may include the same central axis.

In one embodiment, the front chamber has a longer axial length than the rear chamber.

In one embodiment, a lower end of the rear chamber may adjoin an upper portion of the second duct.

In one embodiment, central cross-sections of the front chamber, the rear chamber, the connection duct, the first duct, the second duct, and the third duct may be located in one vertical plane.

### Advantageous Effects

According to embodiments of the present invention, the suction and supply unit can perform supply of an irrigation fluid, suction of blood or other contaminated fluids from a patient's abdominal cavity, or irrigation of the interiors of the front chamber, the rear chamber, the connection duct, and the third duct simply through simple manipulation of the supply valve and the suction valve, thereby ensuring ease of use.

In addition, according to embodiments of the present invention, central cross-sections of the front chamber, the rear chamber, the first duct, the second duct, and the third duct are located in one vertical plane, thereby allowing reduction in thickness of the suction and irrigation device.

It should be understood that advantageous effects of the present invention are not limited thereto and include any advantageous effects conceivable from the features disclosed in the detailed description of the invention or the appended claims.

### DESCRIPTION OF DRAWINGS

FIG. 1 is a perspective view of a vertical-flat-arrangement-type suction and irrigation device according to one embodiment of the present invention.

FIG. 2 is a sectional view of the vertical-flat-arrangement-type suction and irrigation device of FIG. 1, focusing on a housing and a suction and supply unit thereof.

FIG. 3 is an exemplary view illustrating operation of a supply valve of the suction and supply unit of FIG. 2.

FIG. 4 is an exemplary view illustrating operation of a suction valve of the suction and supply unit of FIG. 2.

FIG. 5 is an exploded perspective view of the vertical-flat-arrangement-type suction and irrigation device of FIG. 1, focusing on a holder thereof.

FIG. 6 is a sectional view of the vertical-flat-arrangement-type suction and irrigation device, focusing on the holder of FIG. 5.

### BEST MODE

Reference will now be made in detail to various embodiments, examples of which are illustrated in the accompanying drawings. It should be understood that the present invention may be embodied in different ways and is not limited to the following embodiments. In the drawings, portions irrelevant to the description will be omitted for clarity. Like components will be denoted by like reference numerals throughout the specification.

Throughout the specification, when an element or layer is referred to as being “on,” “connected to,” or “coupled to” another element or layer, it may be directly on, connected to, or coupled to the other element or layer or intervening elements or layers may be present. In addition, unless stated otherwise, the term “includes” should be interpreted as not excluding the presence of other components than those listed herein.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Hereinafter, embodiments of the present invention will be described with reference to the accompanying drawings.

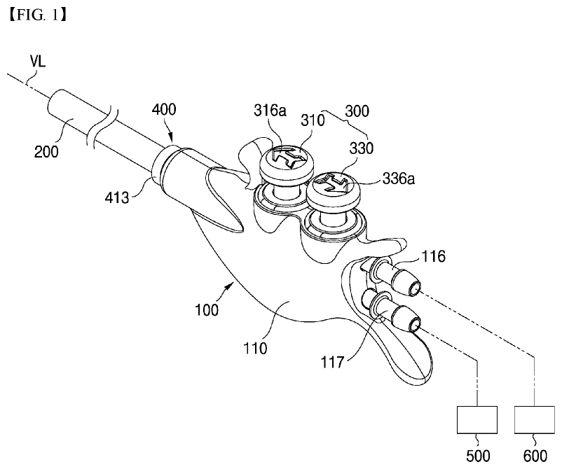

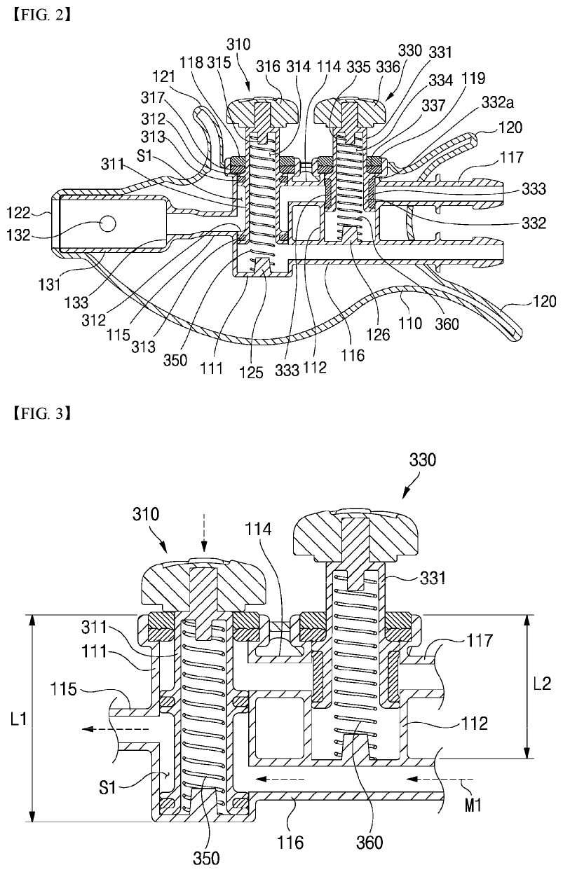

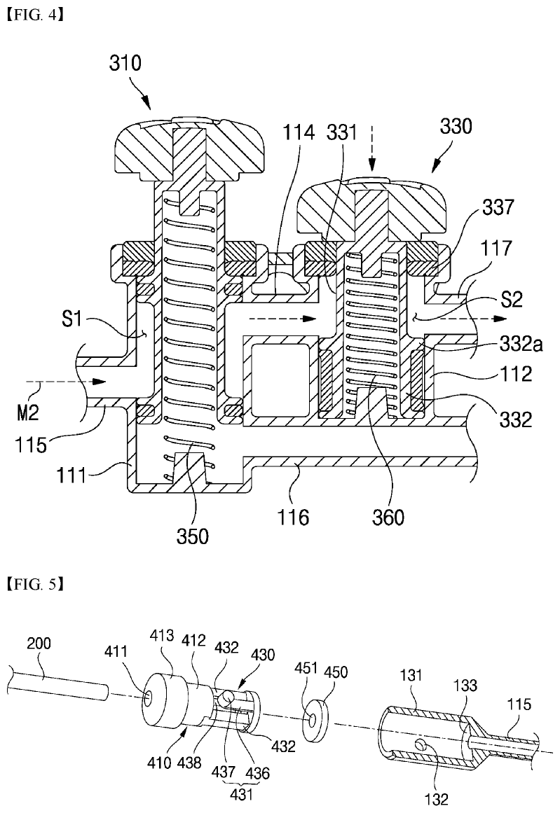

FIG. 1 is a perspective view of a vertical-flat-arrangement-type suction and irrigation device according to one embodiment of the present invention, FIG. 2 is a sectional view of the vertical-flat-arrangement-type suction and irrigation device of FIG. 1, focusing on a housing and a suction and supply unit thereof, FIG. 3 is an exemplary view illustrating operation of a supply valve of the suction and supply unit of FIG. 2, and FIG. 4 is an exemplary view illustrating operation of a suction valve of the suction and supply unit of FIG. 2.

Referring to FIG. 1 to FIG. 4, a vertical-flat-arrangement-type suction and irrigation device may include a housing 100, a probe 200, and a suction and supply unit 300.

For convenience of description, “front” and “rear” will be defined based on the locations of the housing 100 and the probe 200. That is, a direction from the housing 100 to the probe 200 is defined as “front” and a direction from the probe 200 to the housing 100 is defined as “rear”.

The housing 100 may include a front chamber 111, a rear chamber 112, a first duct 115, a second duct 116, and a third duct 117.

Specifically, the housing 100 may include a housing body 110. The housing body 110 may include a first handle 120 and a second handle 121 to provide ease of grip. The first handle 120 may be formed at a rear end of the housing body 110, and the second handle 121 may be formed at an upper front end of the housing body 110.

The front chamber 111 and the rear chamber 112 may be spaced apart from each other, and may be connected to each other via a connection duct 114.

The first duct 115 and the second duct 116 may be connected to the front chamber 111. Specifically, the first duct 115 may be connected to a front of the front chamber 111 to extend forwards, and the second duct 116 may be connected to a rear of the front chamber 111 to extend rearwards. The second duct 116 may be disposed at a lower horizontal position than the first duct 115. The first duct 115 may be disposed inside the housing body 110, and the second duct 116 may extend outwards through the rear end of the housing body 110.

The third duct 117 may be connected to a rear of the rear chamber 112 to extend rearwards, and may be disposed parallel to the second duct 116. The third duct may extend outwards through the rear end of the housing body 110.

Central cross-sections of the front chamber 111, the rear chamber 112, the connection duct 114, the first duct 115, the second duct 116, and the third duct 117 may be located in one vertical plane. Here, the vertical plane may be a vertical plane through which a virtual straight line VL passes. That is, the front chamber 111, the rear chamber 112, the connection duct 114, the first duct 115, the second duct 116, and the third duct 117 may be located in the vertical plane along the virtual straight line VL, thereby allowing reduction in thickness of the vertical-flat-arrangement-type suction and irrigation device. In addition, the front chamber 111, the rear chamber 112, the connection duct 114, the first duct 115, the second duct 116, and the third duct may be integrally formed with one another.

The probe 200 is configured to be inserted into a patient's abdominal cavity, and may be coupled to the first duct 115. Preferably, the probe 200 is detachably coupled to the first duct 115. To this end, the vertical-flat-arrangement-type suction and irrigation device may further include a holder 400, which will be described further below.

The suction and supply unit 300 may include a supply valve 310 and a suction valve 330.

The supply valve 310 may be provided to the front chamber 111. The supply valve 310 may include a supply valve body 311 and a flange 312.

The supply valve body 311 may be partially disposed inside the front chamber 111, and may be reciprocally moved in an axial direction of the front chamber 111. The supply valve body 311 may have an outer peripheral surface spaced apart from an inner peripheral surface of the front chamber 111 such that a space is formed between the supply valve body 311 and the front chamber 111.

The flange 312 may include a pair of flanges protruding from opposite ends of the supply valve body 311 and closely contacting the inner peripheral surface of the front chamber 111. Accordingly, an inner space (referred to as a “front fluid passage”) may be defined by the outer peripheral surface of the supply valve body 311, the inner peripheral surface of the front chamber 111, and the pair of flanges 312. The front fluid passage S1 may be a space formed inside the front chamber 111, and may be moved in conjunction with axial movement of the supply valve 310. The front fluid passage S1 may always remain connected to the first duct 115 regardless of the position of the supply valve body 311.

The supply valve 310 may include a sealing ring 313 disposed on an outer peripheral surface of the flange 312. The sealing ring 313 may closely contact the inner peripheral surface of the front chamber 111, thereby improving sealing at the flange 312 and thus securing fluid-tightness of the front fluid passage S1.

In addition, a first stationary sealing ring 317 may be disposed at an upper portion of the front chamber 111. The first stationary sealing ring 317 may have an outer peripheral surface closely contacting the inner peripheral surface of the front chamber 111 and an inner peripheral surface closely contacting the outer peripheral surface of the supply valve body 311. The first stationary sealing ring 317 may remain stationary regardless of movement of the supply valve body 311. The first stationary sealing ring 317 may provide double leakage prevention in cooperation with the sealing ring 313. A fluid, such as an irrigation fluid and blood or other contaminated fluids from a patient's abdominal cavity, is moved to the front fluid passage S1. When such a fluid leaks out of the front fluid passage S1 through the sealing ring 313, the first stationary sealing ring 317 can prevent further leakage of the fluid. A first cover may be coupled to the upper portion of the front chamber 111. The first cover 118 may secure the first stationary sealing ring 317.

The supply valve body 311 may have a first insertion groove 314 formed therein. The first insertion groove 314 may extend in the axial direction of the supply valve body 311, and may be open outwards at a lower end thereof.

In addition, the supply valve 310 may include a first resilient member 350. The first resilient member 350 may be inserted into the first insertion groove 314 inside the front chamber 111. The first resilient member 350 may have a lower end supported by the front chamber 111 and an upper end supporting an upper portion of the first insertion groove 314, thereby resiliently supporting the supply valve 310 upwards.

The supply valve body 311 may further have a first support protrusion 315 formed at an upper end of the first insertion groove 314 in the axial direction of the supply valve body 311 and disposed inside the first resilient member 350. In addition, the supply valve body 311 may further have a first securing protrusion 125 protruding from a lower end thereof. The first securing protrusion 125 may be inserted into a lower end of the first resilient member 350 to secure the lower end of the first resilient member 350.

For convenience of description, in FIG. 2 to FIG. 5, a moving direction of the supply valve 310 that allows the first resilient member 350 to be compressed is defined as “downward direction” and a moving direction of the supply valve 310 that allows the first resilient member 350 to be extended is defined as “upward direction”.

A first push button 316 may be disposed at the upper end of the supply valve body 311. The first push button 316 may include a first indicator 316 formed on an upper surface thereof and indicating a fluid flow direction upon downward movement of the supply valve 310 by depression of the first push button 316.

The front fluid passage S1 may always remain connected to the first duct 115 regardless of the position of the supply valve 310.

The supply valve 310 may allow the front fluid passage S1 to be connected to the second duct 116 or the connection duct 114 while being reciprocally moved in the axial direction of the front chamber 111. Specifically, when the supply valve 310 is moved upwards by elastic restoring force of the first resilient member 350, the front fluid passage S1 may be connected to the connection duct 114 without being connected to the second duct 116 (see FIG. 2). In addition, when the supply valve 310 is pushed downwards, the front fluid passage S1 may be connected to the second duct without being connected to the connection duct 114 (see FIG. 3).

The suction valve 330 may be provided to the rear chamber 112. Here, a lower end of the rear chamber 112 may adjoin an upper portion of the second duct 116. That is, the rear chamber 112 provided with the suction valve 330 may extend from the upper portion of the second duct 116. An axial length L2 of the rear chamber may be shorter than an axial length L1 of the front chamber 111. Preferably, an upper end of the front chamber 111 is flush with an upper end of the rear chamber 112. In addition, an axial length of the suction valve 330 may be shorter than an axial length of the supply valve 310. Preferably, the upper end of the suction valve 330 is flush with the upper end of the supply valve 310 when the suction valve 330 and the supply valve 310 are not pressed.

The suction valve 330 may include a suction valve body 331 and a sliding block 332. The suction valve body 331 may be partially disposed inside the rear chamber 112, and may be reciprocally moved in the axial direction of the rear chamber 112. An outer peripheral surface of the suction valve body 331 may be spaced apart from an inner peripheral surface of the rear chamber 112. The sliding block 332 may be integrally formed with the suction valve body 331.

In addition, the suction valve 330 may include a sealing plate 333 disposed on the sliding block 332. The sealing plate 333 may be provided in the form of a disc, and may be disposed on both front and rear surfaces of the sliding block 332. The connection duct 114 and the third duct 117 may have the same central axis. Accordingly, when the suction valve 330 is moved, the sealing plate 333 may close the connection duct 114 and the third duct 117 at the same time or may open the connection duct 114 and the third duct 117 at the same time. To this end, the sealing plate 333 may have a larger cross-sectional area than the connection duct 114 and the third duct 117.

In addition, a second stationary sealing ring 337 may be disposed at an upper portion of the rear chamber 112. The second stationary sealing ring 337 may have an outer peripheral surface closely contacting the inner peripheral surface of the rear chamber 112 and an inner peripheral surface closely contacting the outer peripheral surface of the suction valve body 331. The second stationary sealing ring 337 may always remain stationary regardless of movement of the suction valve body 331.

In addition, the suction valve body 331 may have a second insertion groove formed therein. The second insertion groove 334 may extend in the axial direction of the suction valve body 331, and may be open outwards at a lower end thereof.

The suction valve 330 may include a second resilient member 360. The second resilient member 360 may be inserted into the second insertion groove 334 inside the rear chamber 112. The second resilient member 360 may have a lower end supported by the rear chamber 112 and an upper end supporting an upper portion of the second insertion groove 334, thereby resiliently supporting the suction valve 330 upwards.

The suction valve body 331 may further have a second support protrusion formed at the upper end of the second insertion groove 334 in the axial direction of the suction valve body 331 and disposed inside the second resilient member 360. In addition, the suction valve body 331 may have a second securing protrusion 126 protruding from a lower end thereof. The second securing protrusion 126 may be inserted into a lower end of the second resilient member 360 to secure the lower end of the second resilient member 360.

In addition, a second push button 336 may be disposed at an upper end of the suction valve body 331. The second push button 336 may include a second indicator 336a formed on an upper surface thereof and indicating a fluid flow direction upon downward movement of the suction valve 330 by depression of the second push button 336.

When the suction valve body 331 is maintained in an upward position by the second resilient member 360, the sealing plate 333 may close the connection duct 114 and the third duct 117 at the same time (see FIG. 3). When the suction valve body 331 is moved downwards by a user pressing the second push button 336, the sliding block is moved downwards. Then, the sealing plate 333 is also moved downwards such that the connection duct 114 and the third duct 117 are opened (see FIG. 4).

As the sliding block 332 is moved downwards, a rear fluid passage S2 defined by the outer peripheral surface of the suction valve body 331, the inner peripheral surface of the rear chamber 112, the second stationary sealing ring 337, and an upper portion 332 of the sliding block 332 may be formed inside the rear chamber 112. The connection duct 114 and the third duct 117 may be connected to each other by the rear fluid passage S2 (see FIG. 4).

Blood or other contaminated fluids from a patient's abdominal cavity may be moved to the rear fluid passage S2. The second stationary sealing ring 337 prevents leakage of such fluids. In addition, a second cover 119 may be coupled to the upper portion of the rear chamber 112. The second cover 119 may secure the second stationary sealing ring 337.

Referring to FIG. 1, the second duct 116 may be connected to a pump 500 via a tube (not shown). The pump 500 may deliver an irrigation fluid to the second duct 116. The third duct 117 may be connected to a suction unit 600 via a tube (not shown).

As shown in FIG. 2, when the suction valve 330 is in the upward position, the rear fluid passage S2 is not formed in the rear chamber 112. Accordingly, blood or contaminated fluids cannot be drawn from a patient's abdominal cavity despite the presence of suction force delivered to the third duct 117 by the suction unit 600.

In addition, when the supply valve 310 is in the upward position, an irrigation fluid from the pump 500 is not delivered to the front fluid passage S1 despite being delivered to the second fluid passage S2. Accordingly, the irrigation fluid cannot be supplied to a patient's abdominal cavity through the first duct 115 and the probe 200.

As shown in FIG. 3, when the supply valve 310 is moved downwards by depression of the first push button 316 with the suction valve 330 maintained in the upward position, the front fluid passage S1 is connected to the second duct 116. Then, the irrigation fluid M1 delivered to the second duct 116 is moved to the first duct 115 through the front fluid passage S1 to be supplied to a patient through the probe 200. Here, the irrigation fluid M1 in the front fluid passage S1 cannot be moved to the connection duct 114 since the front fluid passage S1 is not connected to the connection duct 114.

In addition, as shown in FIG. 4, when the suction valve 330 is moved downwards by depression of the second push button 336 with the supply valve 310 maintained in the upward position, the rear fluid passage S2 is formed in the rear chamber 112. Here, the rear fluid passage S2 is connected to the front fluid passage S1 and the first duct 115 through the connection duct 114. Accordingly, suction force provided by the suction unit 600 can be delivered to the probe 200 and thus blood or contaminated fluids M2 can be drawn from a patient's abdominal cavity. Here, the blood or contaminated fluids M2 cannot be moved to the second duct 116 since the front fluid passage S1 is not connected to the second duct 116.

The vertical-flat-arrangement-type suction and irrigation device may further include a holder 400.

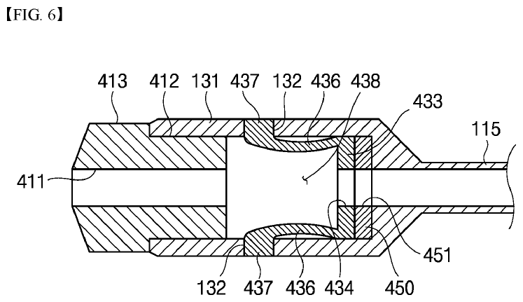

FIG. 5 is an exploded perspective view of the vertical-flat-arrangement-type suction and irrigation device of FIG. 1, focusing on the holder thereof, and FIG. 6 is a sectional view of the vertical-flat-arrangement-type suction and irrigation device, focusing on the holder of FIG. 5.

Referring to FIG. 5 and FIG. 6 along with FIG. 1 and FIG. 2, the holder 400 may be disposed inside the housing body 110, and may be coupled to the first duct 115. The probe 200 may be coupled to the holder 400.

The holder 400 may include a plug 410 and a locking portion 430.

The first duct 115 may include an expansion pipe 131 disposed at a front end thereof. The expansion pipe 131 may have a larger diameter than the first duct 115. The expansion pipe 131 may have a locking hole 132 radially formed therethrough. The locking hole 132 may include multiple locking holes.

The housing body 110 may have an opening 122 formed at the front end thereof. The opening 122 may be connected to the expansion pipe 131.

The plug 410 may have a first through-hole 411 axially formed through a center thereof and allowing the probe 200 to pass therethrough. In addition, the plug may be formed at a rear portion thereof with an insertion portion 412 inserted into the expansion pipe 131, and may be formed at a front portion thereof with a head 413 having a larger diameter than the insertion portion 412 to be stepped with respect to the insertion portion 412, wherein the head 413 is caught on the front end of the expansion pipe 131 upon insertion of the insertion portion 412 into the expansion pipe 131.

The locking portion 430 may be connected to a rear end of the plug 410 to be inserted into the expansion pipe 131, and may include an elastic latch 431 detachably coupled to the locking hole 132. In addition, the locking portion 430 may include a support frame 432 and a pressing plate 433.

The support frame 432 may extend from the rear end of the plug 410 in the axial direction of the expansion pipe 131. The support frame 432 may include multiple support frames spaced apart from one another such that a space 438 is formed inside the multiple support frames.

The pressing plate 433 may be connected to a rear end of the support frame 432. The pressing plate 433 may have a diameter corresponding to the inner diameter of the expansion pipe 131, and may have a second through-hole 434 axially formed through a center thereof and allowing the probe 200 to pass therethrough.

In addition, the holder 400 may include a sealing member 450. The sealing member 450 may be disposed between the pressing plate 433 and a bottom surface of the expansion pipe 131, and may have a third through-hole 451 through which the probe 200 passes. The probe 200 may be inserted into the first duct 115 through the first through-hole 411, the second through-hole 434, and the third through-hole 451. The sealing member 450 may be pressed against the pressing plate 433 and the bottom surface 133 of the expansion pipe 131 so as to prevent an irrigation fluid or blood or contaminated fluids from a patient's abdominal cavity from leaking out of the first duct 115.

The elastic latch 431 may include an elastic bar 436 and a locking protrusion 437.

The elastic bar 436 may have a rear end connected to the pressing plate 433, and may extend toward the plug 410. The elastic bar 436 may be formed between the support frames 432. A space 438 between the elastic bars 436 may provide a place for the elastic bars 436 to be deformed.

The locking protrusion 437 may radially protrude from a front end of the elastic bar 436 and may be coupled to the locking hole 132. The elastic bar 436 may provide elastic force that allows the locking protrusion 437 to be coupled to the locking hole 132. When the elastic bar 436 is deformed toward the space 438 and the locking protrusion 437 is thus released from the locking hole 132, the plug 410 can be separated from the expansion pipe 131.

Although some embodiments have been described herein, it should be understood that these embodiments are provided for illustration only and are not to be construed in any way as limiting the present invention, and that various modifications, changes, alterations, and equivalent embodiments can be made by those skilled in the art without departing from the spirit and scope of the invention. For example, components described as implemented separately may also be implemented in combined form, and vice versa. The scope of the present invention is indicated by the following claims and all changes or modifications derived from the meaning and scope of the claims and equivalents thereto should be construed as being within the scope of the present invention.

### INDUSTRIAL APPLICABILITY

Laparoscopic surgery uses a surgical instrument for supplying an irrigation solution for irrigation of a patient's surgical site and removing contaminated fluids after irrigation of the patient's surgical site through discharge of the contaminated fluids from the patient's body, that is, a laparoscopic suction and irrigation system. As the laparoscopic suction and irrigation system, the vertical-flat-arrangement-type suction and irrigation device according to the present invention is slim and easy to operate and thus can be widely used in the related industry.

1. A vertical-flat-arrangement-type suction and irrigation device comprising:
a housing having a front chamber and a rear chamber spaced apart from each other and connected via a connection duct, a first duct connected to a front of the front chamber and extending forwards, a second duct connected to a rear of the front chamber and extending rearwards, and a third duct connected to a rear of the rear chamber and extending rearwards;
a probe coupled to the first duct and inserted into a patient's abdominal cavity; and
a suction and supply unit having a supply valve reciprocally moved inside the front chamber in an axial direction of the front chamber, the supply valve forming a front fluid passage connected to the first duct and allowing an irrigation fluid to be delivered to the probe through the first duct, and a suction valve reciprocally moved inside the rear chamber in an axial direction of the rear chamber.

2. The vertical-flat-arrangement-type suction and irrigation device according to claim 1, wherein:
the supply valve comprises a supply valve body spaced apart from an inner peripheral surface of the front chamber and a pair of flanges protruding from opposite ends of the supply valve body and closely contacting the inner peripheral surface of the front chamber; and
the front fluid passage is an inner space defined by an outer peripheral surface of the supply valve body, the inner peripheral surface of the front chamber, and the pair of flanges, the front fluid passage being formed inside the front chamber at all times and being moved in conjunction with movement of the supply valve.

3. The vertical-flat-arrangement-type suction and irrigation device according to claim 2, wherein the supply valve further comprises a sealing ring closely contacting outer peripheral surfaces of the pair of flanges and the inner peripheral surface of the front chamber to seal the front fluid passage.

4. The vertical-flat-arrangement-type suction and irrigation device according to claim 2, wherein the supply valve further comprises a first resilient member disposed inside the front chamber and having a lower end supported by the front chamber and an upper end supported by the supply valve to resiliently support the supply valve so as to disconnect the front fluid passage from the second duct.

5. The vertical-flat-arrangement-type suction and irrigation device according to claim 1, wherein the suction valve comprises a suction valve body spaced apart from an inner peripheral surface of the rear chamber, a sliding block protruding from the suction valve body and moved together with the suction valve body, and a stationary sealing ring secured to an upper portion of the rear chamber and closely contacting an outer peripheral surface of the suction valve body, and, when the suction valve is moved downwards, a rear fluid passage defined by the outer peripheral surface of the suction valve body, the inner peripheral surface of the rear chamber, the stationary sealing ring, and an upper portion of the sliding block is formed, the rear fluid passage being connected to the connection duct and the third duct.

6. The vertical-flat-arrangement-type suction and irrigation device according to claim 5, wherein the suction valve further comprises a sealing plate disposed on the sliding block and closely contacting the inner peripheral surface of the rear chamber, the sealing plate closing the connection duct and the third duct upon upward movement of the suction valve body while opening the connection duct and the third duct upon downward movement of the suction valve body.

7. The vertical-flat-arrangement-type suction and irrigation device according to claim 6, wherein the suction valve further comprises a second resilient member disposed inside the rear chamber and having a lower end supported by the rear chamber and an upper end supported by the suction valve to resiliently support the suction valve such that the connection duct and the third duct are closed by the sealing plate.

8. The vertical-flat-arrangement-type suction and irrigation device according to claim 6, wherein the connection duct and the third duct have the same central axis.

9. The vertical-flat-arrangement-type suction and irrigation device according to claim 1, wherein the front chamber has a longer axial length than the rear chamber.

10. The vertical-flat-arrangement-type suction and irrigation device according to claim 1, wherein a lower end of the rear chamber adjoins an upper portion of the second duct.

11. The vertical-flat-arrangement-type suction and irrigation device according to claim 1, wherein central cross-sections of the front chamber, the rear chamber, the connection duct, the first duct, the second duct, and the third duct are located in one vertical plane.
